(19) United States
(12) Patent Application Publication
(10) Pub. No.: US 2023/0301615 A1
(43) Pub. Date: Sep. 28, 2023

(54) RADIATION IMAGING SYSTEM, RADIATION IMAGING APPARATUS, CONTROL METHOD, AND STORAGE MEDIUM

(71) Applicant: CANON KABUSHIKI KAISHA, Tokyo (JP)

(72) Inventors: SHIGEO KOBAYASHI, Kanagawa (JP); JUNYA ODORI, Kanagawa (JP); YUTAKA ISHINARI, Kanagawa (JP)

(21) Appl. No.: 18/187,113

(22) Filed: Mar. 21, 2023

(30) Foreign Application Priority Data
Mar. 28, 2022 (JP) 2022-051871

(51) Int. Cl. A61B 6/00

(52) U.S. Cl. CPC A61B 6/563

(57) ABSTRACT

A radiation imaging system includes an access point that performs first wireless communication with a radiation imaging apparatus that performs imaging to acquire a radiographic image, the first wireless communication being performed to control the imaging by the radiation imaging apparatus, a communication device that performs second wireless communication with the radiation imaging apparatus to make a setting to be used for the first wireless communication on the radiation imaging apparatus, and a control apparatus that controls the imaging. The control apparatus causes the communication device to perform the second wireless communication with the radiation imaging apparatus based on signal intensity of a signal received from the radiation imaging apparatus by the communication device and a predetermined number of times that the signal intensity exceeds a predetermined threshold.

### BACKGROUND

### Field

The present disclosure relates to a radiation imaging system, a radiation imaging apparatus, a control method, and a storage medium.

### Description of the Related Art

In the medical field, radiation imaging systems using radiation are known. The digitalization of the radiation imaging systems enables the widespread use of a system in which a radiation generation apparatus irradiates a radiation imaging apparatus with radiation through a subject, the radiation imaging apparatus generates a digital radiographic image, and an imaging control apparatus enables viewing the image immediately after the radiation imaging. This enables offering an improved workflow and performing imaging with a fast cycle compared to a conventional imaging method using a film.

In such a radiation imaging system, a radiation imaging apparatus and an imaging control apparatus that are wirelessly connected to eliminate installation restrictions due to a cable of the radiation imaging apparatus are discussed. To establish the wireless connection between these apparatuses, it is necessary to use the same settings, such as a service set identifier (SSID), an authentication method, an encryption type, and an encryption key, between the apparatuses to be connected. It is common that these settings are manually set on both the apparatuses to be wirelessly connected, or are set using a push button method or a personal identification number (PIN) code method defined by Wi-Fi Protected Setup™ (WPS).

In a case where the settings are manually made, an input operation is required, and establishment of the connection can fail because of an operational error. Similarly, the PIN code method requires an operation of inputting a PIN cord to a master apparatus. The push button method requires an operation different from an operation in a normal workflow of the radiation imaging system, such as pressing or touching both of a push button of a slave apparatus and a push button of a master apparatus at the same time.

Japanese Patent Application Laid-Open No. 2011-120885 discusses a radiation imaging system including a unit that configures wireless settings between a radiation imaging apparatus and an imaging control apparatus via a connection unit that uses short-range wireless communication, such as infrared communication or Bluetooth® communication, having a communication range smaller than the range of wireless communication. This eliminates the need for an operator to manually set the wireless settings. With the method according to Japanese Patent Application Laid-Open No. 2011-120885, in a case where another radiation imaging system is nearby, settings for connecting to an unintended wireless apparatus can be set on the radiation imaging apparatus.

### SUMMARY

The present disclosure is directed to a system in which settings for connecting to an intended wireless apparatus can be easily and accurately set on a radiation imaging apparatus.

According to an aspect of the present disclosure, a radiation imaging system includes an access point configured to perform first wireless communication with a radiation imaging apparatus configured to perform imaging to acquire a radiographic image, the first wireless communication being performed to control the imaging by the radiation imaging apparatus, a communication device configured to perform second wireless communication with the radiation imaging apparatus to make a setting to be used for the first wireless communication on the radiation imaging apparatus, and a control apparatus configured to control the imaging. The control apparatus causes the communication device to perform the second wireless communication with the radiation imaging apparatus based on signal intensity of a signal received from the radiation imaging apparatus by the communication device and a predetermined number of times that the signal intensity exceeds a predetermined threshold.

Further features will become apparent from the following description of exemplary embodiments with reference to the attached drawings.

### BRIEF DESCRIPTION OF THE DRAWINGS

FIG. 1 is a block diagram illustrating an overall configuration of a radiation imaging system.

FIG. 2 is a block diagram illustrating a configuration of a main part of a radiation imaging apparatus.

FIG. 3 is a flowchart illustrating a connection operation according to a first exemplary embodiment.

FIG. 4 is a sequence diagram illustrating connection control according to the first exemplary embodiment.

FIG. 5 is a diagram illustrating an example of arrangement of apparatuses according to the first exemplary embodiment.

FIGS. 6A and 6B are tables schematically illustrating detection conditions and detection results, respectively, according to the first exemplary embodiment.

FIG. 7 is a table schematically illustrating detection conditions according to a second exemplary embodiment.

FIG. 8 is a flowchart illustrating a connection operation according to the second exemplary embodiment.

FIG. 9 is a diagram illustrating an example of arrangement of apparatuses according to the second exemplary embodiment.

FIGS. 10A and 10B are tables each schematically illustrating detection results according to the second exemplary embodiment.

### DESCRIPTION OF THE EMBODIMENTS

Exemplary embodiments of the present disclosure will be described below with reference to the attached drawings. The following exemplary embodiments are not intended to be limiting. Not all of combinations of features described in the exemplary embodiments are essential to solving means of the disclosure.

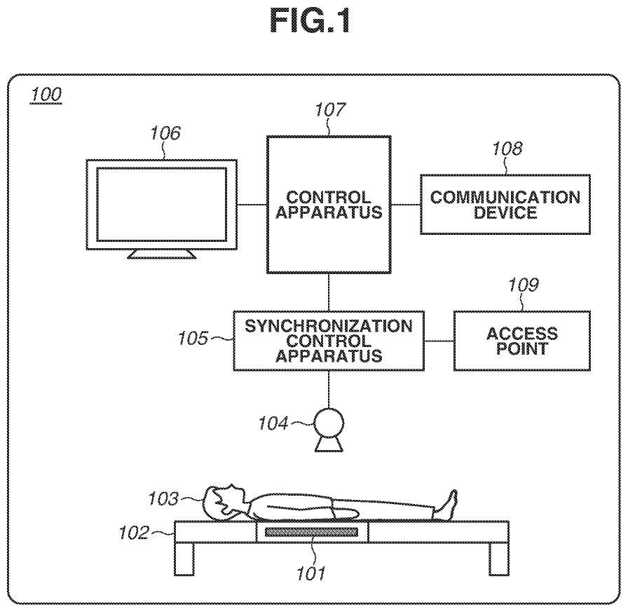

A radiation imaging system according to a first exemplary embodiment of the present disclosure will be described with reference to FIGS. 1 to 6B.

FIG. 1 is a block diagram illustrating an overall configuration of a radiation imaging system 100 according to the present exemplary embodiment.

The radiation imaging system 100 includes a radiation imaging apparatus 101, a lying position type imaging support 102, a radiation generation apparatus 104, a synchronization control apparatus 105, a display apparatus 106, a control apparatus 107, an access point 109, and a communication device 108.

The radiation imaging apparatus 101 is mounted in the lying position type imaging support 102, and acquires a radiographic image based on radiation applied downward to a subject 103 by the radiation generation apparatus 104. In the example of FIG. 1, the radiation imaging apparatus 101 performs imaging while being mounted in the lying position type imaging support 102, but the radiation imaging apparatus 101 can perform imaging in any other way. For example, the radiation imaging apparatus 101 can perform imaging while being mounted in a standing position type imaging support, or can perform imaging without using an imaging support.

The control apparatus 107 performs operations such as displaying a radiographic image captured by the radiation imaging apparatus 101 on the display apparatus 106, and issuing an instruction about imaging conditions input via an operation unit. The control apparatus 107 also transmits setting information for enabling wireless communication between the radiation imaging apparatus 101 and the control apparatus 107.

The access point 109 is a radio wave relay device that wirelessly exchanges information with the radiation imaging apparatus 101.

The communication device 108 is a radio wave transmitter/receiver for performing short-range communication between the radiation imaging apparatus 101 and the control apparatus 107. The communication device 108 can be included in the control apparatus 107. For example, the communication device 108 is a dongle connected to the control apparatus 107 via a Universal Serial Bus (USB) interface.

Bluetooth® is desirably used for the communication device 108, but a device implemented by any other known communication technique can be used as long as the device performs short-range wireless communication. For example, the communication device 108 can be a device supporting the Bluetooth® Basic Rate/Enhanced Data Rate standard or the Bluetooth® Low Energy standard. The communication device 108 can also be a Radio Frequency Identifier (RFID) device that exchanges information with a tag in which ID information is embedded, by short-range wireless communication using an electromagnetic field, radio waves, or the like. The communication device 108 can have the function of the access point 109.

The communication method of the RFID can be either an electromagnetic induction method or a radio wave method. A function incorporated in another apparatus such as the radiation generation apparatus 104 can alternatively be used as the function of the communication device 108.

The synchronization control apparatus 105 includes a circuit mediating communication, and monitors the state of the radiation imaging apparatus 101 and the radiation generation apparatus 104. For example, the synchronization control apparatus 105 controls the irradiation of radiation from the radiation generation apparatus 104 and the imaging of the subject 103 by the radiation imaging apparatus 101. The synchronization control apparatus 105 can include a built-in hub for connecting a plurality of network devices.

The radiation generation apparatus 104 includes a radiation tube that causes electrons to hit an anode by accelerating the electrons at a high voltage in order to generate radiation such as X rays. The radiation can be α rays, β rays, γ rays, X rays, or neutron rays.

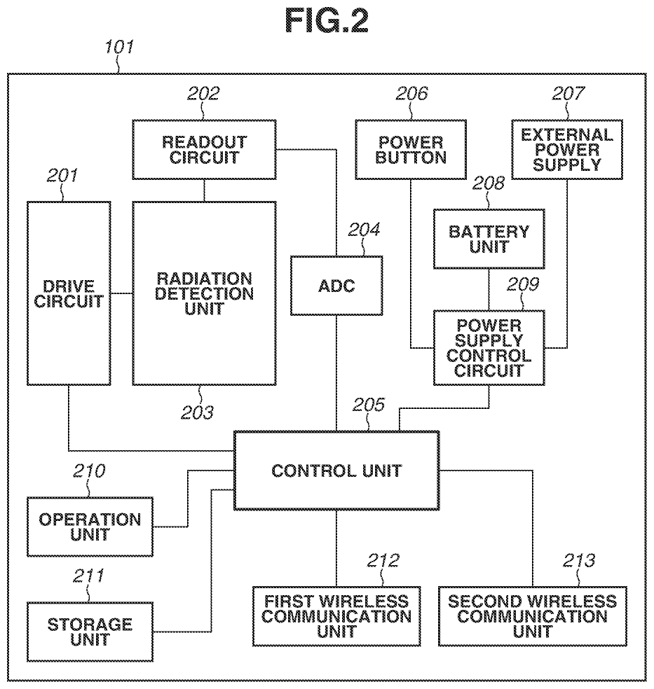

FIG. 2 is a schematic block diagram illustrating a configuration of a main part of the radiation imaging apparatus 101.

A power button 206 is an operation unit for starting or stopping power supply to each component of the radiation imaging apparatus 101. Any implementation of the operation unit that would enable starting or stopping power supply is applicable. A user prepares for imaging by operating the power button 206.

A battery unit 208 supplies a predetermined voltage to each component of the radiation imaging apparatus 101. Any known type of battery can be used, such as a lithium-ion battery or an electric double layer capacitor. In a case where power is supplied from an external power supply 207 (described below) to the radiation imaging apparatus 101, the battery unit 208 can be excluded.

The external power supply 207 supplies a predetermined voltage from an external power source to the radiation imaging apparatus 101. The external power supply 207 can supply power in a wired manner or in a wireless, non-contact manner.

A power supply control circuit 209 controls power to be supplied from the battery unit 208 or the external power supply 207 to each component of the radiation imaging apparatus 101, depending on an operating state of the power button 206, and monitors a battery remaining amount. For example, the power supply control circuit 209 transforms a voltage from the battery unit 208 into a predetermined voltage, and supplies the predetermined voltage to each component. For example, in a case where the external power supply 207 is not connected to the radiation imaging apparatus 101, the power supply from the battery unit 208 can be turned on or off by pressing the power button 206.

A radiation detection unit 203 detects radiation having passed through the subject 103 as image signals (electric charges). For example, the radiation detection unit 203 includes a scintillator that converts the radiation into light, and a photoelectric conversion element that converts the light generated by the scintillator into image signals (electric charges) that are electrical signals.

A drive circuit 201 is an integrated circuit (IC) that applies a drive signal to the radiation detection unit 203, and performs operations such as accumulation and readout of the image signals (electric charges). A readout circuit 202 has a function of amplifying the image signals (electric charges) output to a signal line, and sequentially reads out the image signals of the radiation detection unit 203. An analog-to-digital (A/D) converter (ADC) 204 converts the analog image signals read out by the readout circuit 202 into digital image signals, and outputs the digital image signals to a control unit 205 as a radiographic image. In other words, the ADC 204 is an A/D conversion unit that converts the analog image signals read out by the readout circuit 202 into digital data.

A storage unit 211 stores radiographic image data output from the ADC 204, a system identifier, a distance threshold calculated from signal intensity between the radiation imaging apparatus 101 and the communication device 108, and an offset image. The storage unit 211 can also store a technician ID that is identification information about a technician corresponding to generated image data, a patient ID that is identification information about a patient, imaging conditions including an imaging time, an imaging dose, an imaging region, and the number of captured images, and a transfer history of the radiographic image data, in association with each other.

The storage unit 211 is a readable and writable device, for example, a nonvolatile memory such as a flash memory. The storage unit 211 is not limited thereto and can be a volatile storage device such as a synchronous dynamic random access memory (SDRAM). The storage unit 211 can be a detachable device such as a secure digital (SD) memory card, and be attached to the control apparatus 107 or the like.

A first wireless communication unit 212 is a wireless communication module supporting a medium to be used for communication with the control apparatus 107 and the synchronization control apparatus 105. For example, the first wireless communication unit 212 can communicate with the access point 109 using a wireless local area network (WLAN) to transmit and receive data such as a radiographic image to and from the control apparatus 107.

A second wireless communication unit 213 is a wireless communication module supporting a medium to be used for communication with the control apparatus 107 and the synchronization control apparatus 105. For example, the second wireless communication unit 213 communicates with the communication device 108 using a wireless personal area network (WPAN). The second wireless communication unit 213 can transmit an identifier of the radiation imaging system 100 and receive wireless communication settings for communication with the first wireless communication unit 212, such as a service set identifier (SSID), an encryption key, and an Internet Protocol (IP) address.

In the present exemplary embodiment, the second wireless communication unit 213 functions as a reception unit that receives setting information transmitted from the communication device 108 and to be used for wireless communication between the first wireless communication unit 212 and the access point 109.

An operation unit 210 can be used as a manual trigger for exchanging setting information between the radiation imaging apparatus 101 and the communication device 108. For example, operating the operation unit 210 enables exchanging the identifier of the radiation imaging system 100, antenna information to be set for the first wireless communication unit 212, the SSID, the encryption key, the IP address, and the like.

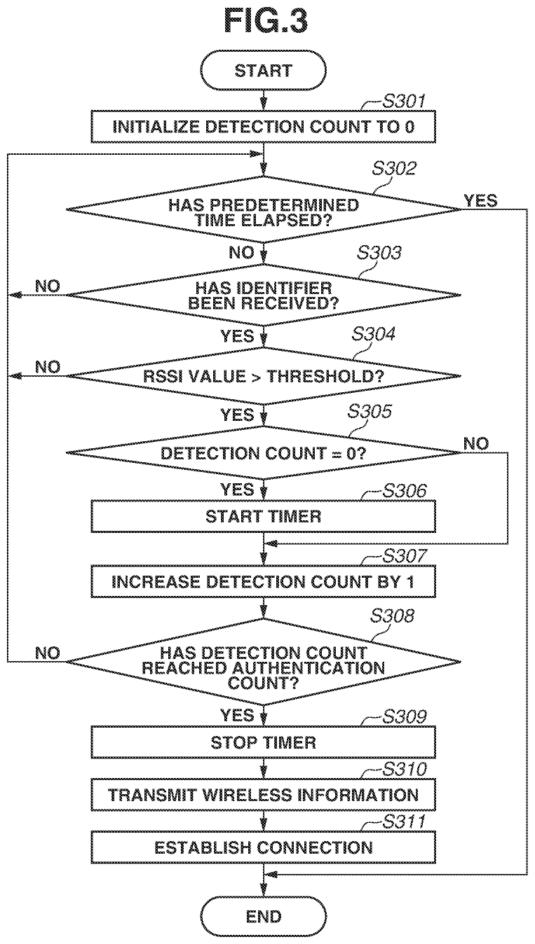

Connection control performed by the control apparatus 107 to establish a connection between the access point 109 and the first wireless communication unit 212 will be described with reference to FIG. 3. FIG. 3 is a flowchart illustrating a connection operation for establishing the connection.

In step S301, the control apparatus 107 initializes the number of times that the communication device 108 has detected the identifier transmitted from the second wireless communication unit 213 (which is also referred to as a detection count) to 0. In step S302, the control apparatus 107 determines whether a predetermined time has elapsed since the first detection of the identifier. In a case where the predetermined time has elapsed (YES in step S302), the processing ends. In a case where the predetermined time has not elapsed (NO in step S302), the processing proceeds to step S303.

In step S303, the control apparatus 107 determines whether the communication device 108 has received the identifier from the second wireless communication unit 213. In a case where the identifier has not been received (NO in step S303), the processing returns to step S302. In a case where the identifier has been received (YES in step S303), the processing proceeds to step S304.

In step S304, the control apparatus 107 determines whether signal intensity (or received signal strength indicator (RSSI) value) of the signal of the identifier received in step S303 exceeds a predetermined threshold. In a case where the predetermined threshold is not exceeded (NO in step S304), the processing returns to step S302. In a case where the predetermined threshold is exceeded (YES in step S304), the processing proceeds to step S305.

In step S305, the control apparatus 107 determines whether the detection count is 0. In a case where the detection count is 0 (YES in step S305), the processing proceeds to step S306. In step S306, the control apparatus 107 starts a timer for measuring a lapse of time after the first detection of the identifier, and the processing proceeds to step S307. In a case where the detection count is not 0 (NO in step S305), the processing proceeds to step S307. In step S307, the control apparatus 107 increases the detection count by one.

In step S308, the control apparatus 107 determines whether the detection count has reached a predetermined authentication count.

In a case where the predetermined authentication count is not reached (NO in step S308), the processing returns to step S302. In a case where the predetermined authentication count is reached (YES in step S308), the processing proceeds to step S309. In step S309, the control apparatus 107 stops the timer started in step S306, and the processing proceeds to step S310.

In step S310, the control apparatus 107 establishes a connection between the communication device 108 and the second wireless communication unit 213, and terminates the connection upon transmission of wireless information from the communication device 108 to the second wireless communication unit 213. The processing then proceeds to step S311. In step S311, the control apparatus 107 establishes a connection between the access point 109 and the first wireless communication unit 212, and the processing ends.

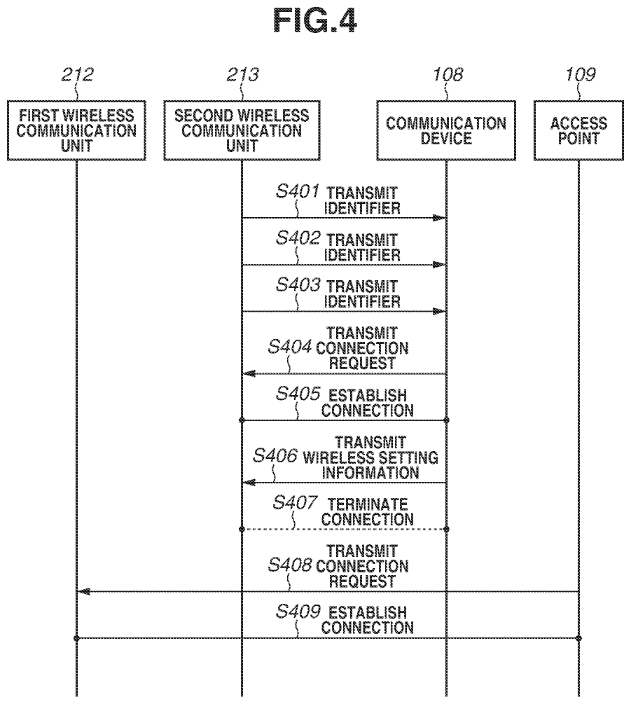

Control to establish the connection between the access point 109 and the first wireless communication unit 212 will be described with reference to FIG. 4. FIG. 4 is a sequence diagram illustrating connection control to establish the connection.

In steps S401, S402, and S403, the identifier is transmitted from the second wireless communication unit 213 to the communication device 108. In step S404, after the number of times that the identifier has been detected has reached the predetermined authentication count, a connection request is transmitted from the communication device 108 to the second wireless communication unit 213.

In step S405, the connection between the communication device 108 and the second wireless communication unit 213 is established. In step S406, wireless setting information is transmitted from the communication device 108 to the second wireless communication unit 213. In step S407, the connection between the communication device 108 and the second wireless communication unit 213 is terminated. In step S408, a connection request is transmitted from the access point 109 to the first wireless communication unit 212. In step S409, the connection between the access point 109 and the first wireless communication unit 212 is established, and the control ends.

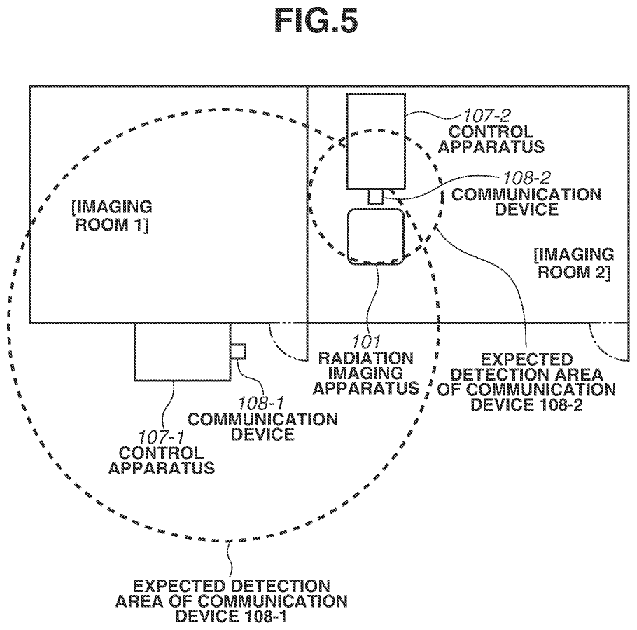

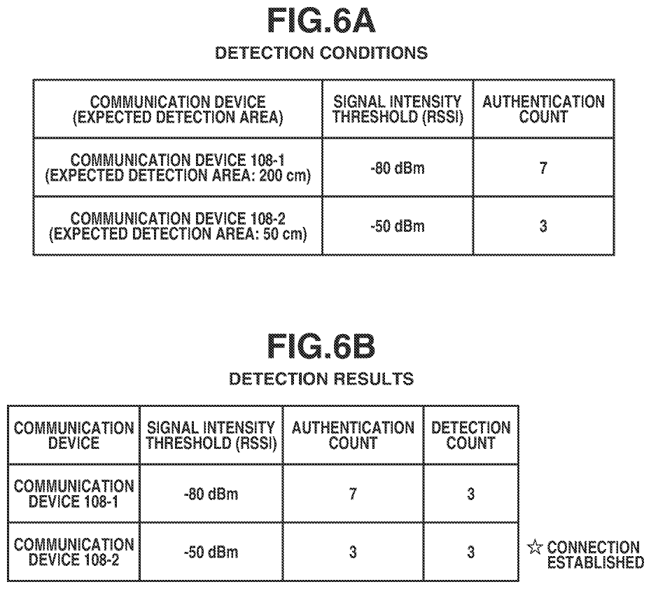

Next, a method for connecting the radiation imaging apparatus 101 and the control apparatus 107 in a case where a plurality of control apparatus systems is in proximity to each other will be described with reference to FIGS. 5, 6A, and 6B. FIG. 5 illustrates an example of arranging the two control apparatuses 107 (107-1 and 107-2) in two adjacent imaging rooms 1 and 2. FIG. 6A is a table illustrating detection conditions for the communication devices 108 (108-1 and 108-2) respectively connected to the control apparatuses 107-1 and 107-2 to detect signals from the second wireless communication unit 213.

In the use condition in FIG. 5, the control apparatus 107-1 is expected to connect to the radiation imaging apparatus 101 in a wide area inside the imaging room 1, and the control apparatus 107-2 is expected to connect to the radiation imaging apparatus 101 at a short distance inside the imaging room 2. The table of FIG. 6A indicates that an expected detection area of the communication device 108-1 is 200 cm, and an expected detection area of the communication device 108-2 is 50 cm. Each of the detection areas is indicated by a dotted circle in FIG. 5.

The distance and the signal intensity between the radiation imaging apparatus 101 and the communication device 108 have a different relationship depending on the output of a wireless module, and thus it is desirable that the relationship between the distance and the signal intensity be measured for each operating environment to be used and a signal intensity threshold be set.

The signal intensity typically weakens as the distance increases. In an actual operating environment, however, the variation in value becomes larger as the distance increases due to, for example, the influence of reflected waves. Thus, in the table of FIG. 6A, a greater signal intensity threshold is set for the communication device 108-2 than the communication device 108-1. A greater authentication count is set for the communication device 108-1 than the communication device 108-2 in order to prevent false detection due to the variation in signal intensity.

Even in a case where the radiation imaging apparatus 101 in the imaging room 2 is within both of the expected detection areas of the communication devices 108-1 and 108-2 indicated by the dotted circles in FIG. 5, setting the detection conditions as illustrated in FIG. 6A enables the radiation imaging apparatus 101 to establish a connection with the communication device 108-2 having a less authentication count after the start of transmission of the identifier from the radiation imaging apparatus 101, as illustrated in FIG. 6B.

In this way, the signal intensity threshold and the authentication count are set for the control apparatuses 107-1 and 107-2 depending on the use condition, so that an unintended connection between the radiation imaging apparatus 101 and one of the control apparatuses 107-1 or 107-2 can be prevented. The signal intensity threshold and the authentication count can be determined depending on the distance of the expected detection area, or can be set individually.

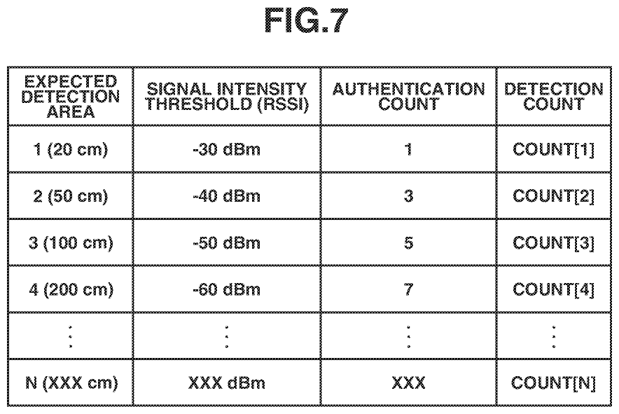

Next, a second exemplary embodiment of the present disclosure will be described with reference to FIGS. 7 to 10B.

FIG. 7 is a table illustrating detection conditions for the communication device 108 to detect a signal from the second wireless communication unit 213 according to the second exemplary embodiment. The table of FIG. 7 indicates that the control apparatus 107 has an N-number of detection conditions, has a signal intensity threshold and an authentication count that are settable for each expected detection area, and stores the detection count in each of the N-number of detection conditions as “count[ 1 to N]”.

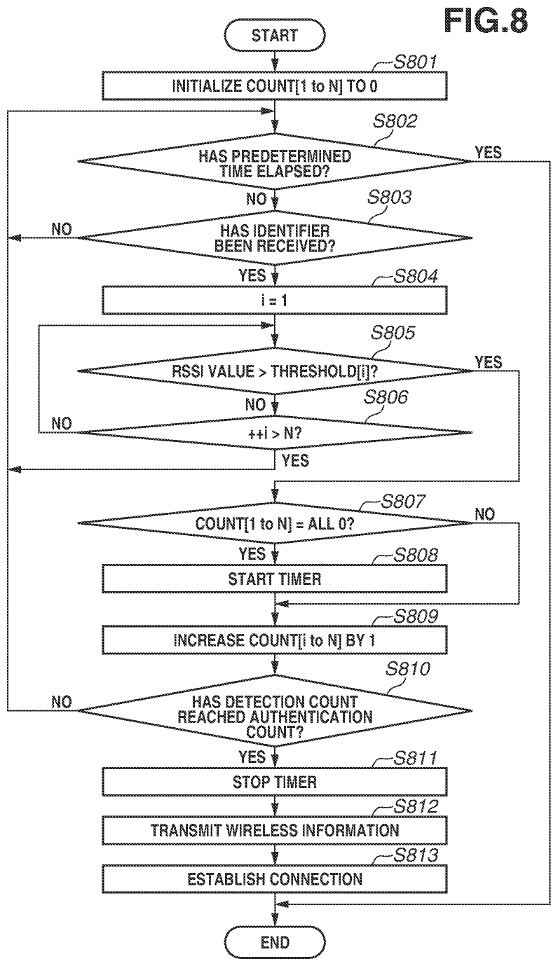

Processing performed by the control apparatus 107 to establish the connection between the access point 109 and the first wireless communication unit 212 will be described with reference to FIG. 8. FIG. 8 is a flowchart illustrating a connection operation performed by the control apparatus 107 to establish the connection.

In step S801, the control apparatus 107 initializes the number of times that the communication device 108 has detected the identifier transmitted from the second wireless communication unit 213, i.e., “count[1 to N]” to 0. In step S802, the control apparatus 107 determines whether a predetermined time has elapsed since the first detection of the identifier. In a case where the predetermined time has elapsed (YES in step S802), the processing ends. In a case where the predetermined time has not elapsed (NO in step S802), the processing proceeds to step S803.

In step S803, the control apparatus 107 determines whether the communication device 108 has received the identifier from the second wireless communication unit 213. In a case where the identifier has not been received (NO in step S803), the processing returns to step S802. In a case where the identifier has been received (YES in step S803), the processing proceeds to step S804.

In step S804, the control apparatus 107 sets a counter i for confirming the N-number of detection conditions to 1. In step S805, the control apparatus 107 determines whether the signal intensity (or RSSI value) of the signal of the identifier received in step S803 exceeds a predetermined threshold[i]. In a case where the predetermined threshold[i] is not exceeded (NO in step S805), the processing proceeds to step S806.

In step S806, the control apparatus 107 increments the counter i by one and then determines whether the counter i exceeds N. In a case where the counter i exceeds N (YES in step S806), the processing returns to step S802. In a case where the counter i does not exceed N (NO in step S806), the processing returns to step S805. In a case where the predetermined threshold[i] is exceeded (YES in step S805), the processing proceeds to step S807.

In step S807, the control apparatus 107 determines whether the detection counts “count[1 to N]” are all 0. In a case where the detection counts “count[1 to N]” are all 0 (YES in step S807), the processing proceeds to step S808. In step S808, the control apparatus 107 starts a timer for measuring a lapse of time after the first detection of the identifier, and the processing proceeds to step S809. In a case where the detection counts “count[1 to N]” are not all 0 (NO in step S807), the processing proceeds to step S809.

In step S809, the control apparatus 107 increases the detection count “count[i to N]” by one. In step S810, the control apparatus 107 determines whether the detection count “count[1 to N]” has reached a predetermined authentication count. In a case where the predetermined authentication count is not reached (NO in step S810), the processing returns to step S802. In a case where the predetermined authentication count is reached (YES in step S810), the processing proceeds to step S811.

In step S811, the control apparatus 107 stops the timer started in step S808, and the processing proceeds to step S812. In step S812, the control apparatus 107 establishes the connection between the communication device 108 and the second wireless communication unit 213, and terminates the connection upon transmission of wireless information from the communication device 108 to the second wireless communication unit 213. The processing then proceeds to step S813. In step S813, the control apparatus 107 establishes the connection between the access point 109 and the first wireless communication unit 212, and the processing ends.

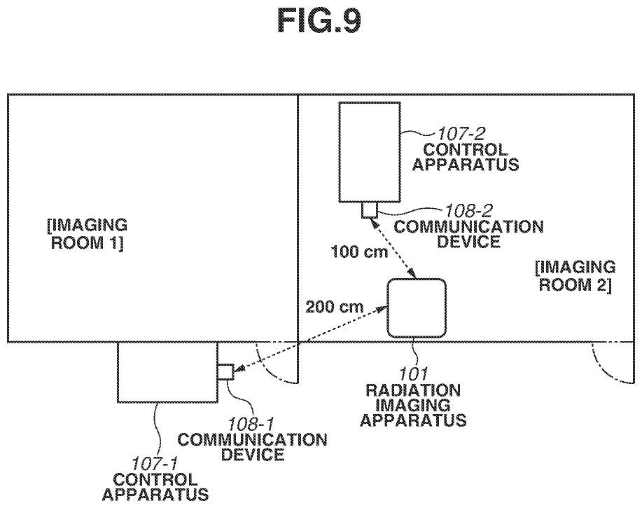

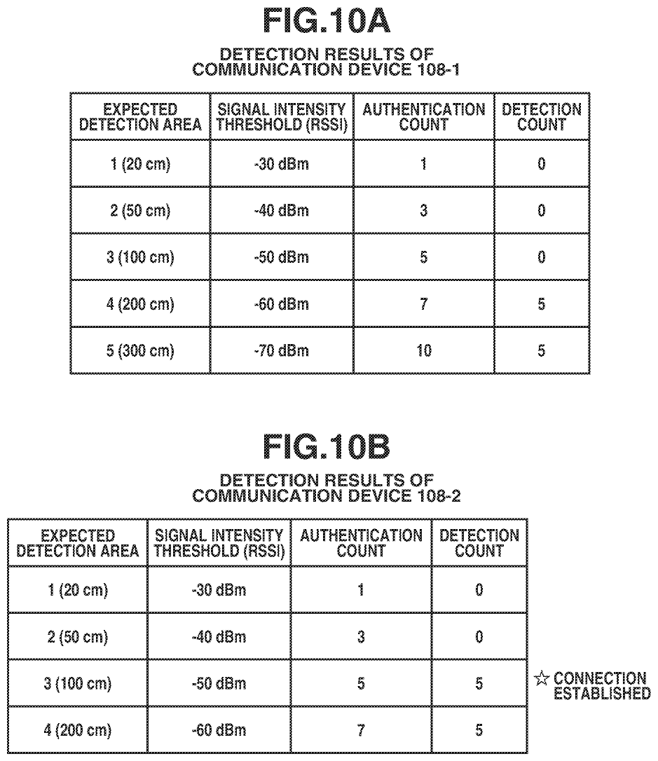

Next, a method for connecting the radiation imaging apparatus 101 and the control apparatus 107 in a case where a plurality of control apparatus systems is in proximity to each other will be described with reference to FIGS. 9, 10A, and 10B. FIG. 9 illustrates an example of arranging the two control apparatuses 107 (107-1 and 107-2) in two adjacent imaging rooms 1 and 2. FIGS. 10A and 10B are tables illustrating detection conditions of the control apparatuses 107-1 and 107-2 and detection results of the communication apparatuses 108-1 and 108-2, respectively.

In the example of FIG. 9, the control apparatuses 107-1 and 107-2 are arranged so that the distance between the communication device 108-1 and the radiation imaging apparatus 101 is 200 cm, and the distance between the communication device 108-2 and the radiation imaging apparatus 101 is 100 cm.

FIGS. 10A and 10B illustrate an example in which the control apparatus 107-1 stores five detection conditions with a maximum expected detection area of 300 cm, and the control apparatus 107-2 stores four detection conditions with a maximum expected detection area of 200 cm.

FIG. 10A illustrates the detection results of the communication device 108-1. When the identifier is transmitted from the radiation imaging apparatus 101, a signal satisfying the signal intensity threshold for each of the expected detection areas of 200 cm and 300 cm is detected, so that the detection count in each of the detection areas is increased each time the identifier is detected.

FIG. 10B illustrates the detection results of the communication device 108-2. When the identifier is transmitted from the radiation imaging apparatus 101, a signal satisfying the signal intensity threshold for each of the expected detection areas of 100 cm and 200 cm is detected, so that the detection count in each of the detection areas is increased each time the identifier is detected. After the start of transmission of the identifier from the radiation imaging apparatus 101, the detection count in the expected detection area of 100 cm of the communication device 108-2 reaches the authentication count faster than the others, and thus a connection between the radiation imaging apparatus 101 and the communication device 108-2 at a short distance therefrom is established.

As described above, a plurality of signal intensity thresholds and a plurality of authentication counts are set for each radiation imaging apparatus depending on the use environment. This enables providing a radiation imaging system that prevents an unintended connection between an imaging apparatus and a control apparatus and also enables a connection between the imaging apparatus and the control apparatus in a wide detection area.

### OTHER EMBODIMENTS

Embodiment(s) can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While exemplary embodiments have been described, these embodiments are not seen to be limiting. The scope of the following claims is to be accorded the broadest interpretation so as to encompass all such modifications and equivalent structures and functions.

This application claims the benefit of Japanese Patent Application No. 2022-051871, filed Mar. 28, 2022, which is hereby incorporated by reference herein in its entirety.

What is claimed is:

1. A radiation imaging system comprising:
an access point configured to perform first wireless communication with a radiation imaging apparatus configured to perform imaging to acquire a radiographic image, the first wireless communication being performed to control the imaging by the radiation imaging apparatus;
a communication device configured to perform second wireless communication with the radiation imaging apparatus to make a setting to be used for the first wireless communication on the radiation imaging apparatus; and
a control apparatus configured to control the imaging,
wherein the control apparatus causes the communication device to perform the second wireless communication with the radiation imaging apparatus based on signal intensity of a signal received from the radiation imaging apparatus by the communication device and a predetermined number of times that the signal intensity exceeds a predetermined threshold.

2. The radiation imaging system according to claim 1, wherein the predetermined threshold is set based on a distance between the communication device and the radiation imaging apparatus.

3. The radiation imaging system according to claim 1, wherein the predetermined number of times is set based on the signal intensity.

4. The radiation imaging system according to claim 1, wherein the control apparatus causes the communication device to perform the second wireless communication with the radiation imaging apparatus in a case where the signal intensity exceeds the predetermined threshold the predetermined number of times.

5. The radiation imaging system according to claim 1, wherein the radiation imaging apparatus includes a first wireless communication unit configured to perform the first wireless communication and a second wireless communication unit configured to perform the second wireless communication.

6. The radiation imaging system according to claim 5, wherein the first wireless communication unit performs the first wireless communication with the access point using a wireless local area network (WLAN) and the second wireless communication unit performs the second wireless communication with the communication device using a wireless personal area network (WPAN).

7. The radiation imaging system according to claim 6, wherein the communication device and the second wireless communication unit support one or more of the Bluetooth® Basic Rate/Enhanced Data Rate standard or the Bluetooth® Low Energy standard.

8. The radiation imaging system according to claim 1,
wherein the radiation imaging system comprises a plurality of the control apparatuses, and
wherein the predetermined threshold and the predetermined number of times are set for each of the plurality of control apparatuses.

9. A radiation imaging apparatus configured to perform imaging to acquire a radiographic image, the radiation imaging apparatus comprising:
a first wireless communication unit configured to perform first wireless communication with an access point of a radiation imaging system to control the imaging;
a second wireless communication unit configured to perform second wireless communication with a communication device of the radiation imaging system to make a setting to be used for the first wireless communication; and
a reception unit configured to receive, using the second wireless communication unit, setting information for making the setting, the setting information being transmitted from the communication device based on a predetermined number of times that signal intensity between the radiation imaging apparatus and the communication device exceeds a predetermined threshold.

10. A method for controlling a radiation imaging system including an access point configured to perform first wireless communication with a radiation imaging apparatus configured to perform imaging to acquire a radiographic image, the first wireless communication being performed to control the imaging by the radiation imaging apparatus, and a communication device configured to perform second wireless communication with the radiation imaging apparatus to make a setting to be used for the first wireless communication on the radiation imaging apparatus, the method comprising:
performing a first determination whether signal intensity between the communication device and the radiation imaging apparatus exceeds a predetermined threshold; and
performing a second determination whether to perform the second wireless communication with the radiation imaging apparatus based on a predetermined number of times that the signal intensity exceeds the predetermined threshold in the first determination.

11. A non-transitory computer-readable storage medium storing a program for causing a computer to execute a method for controlling a radiation imaging system including an access point configured to perform first wireless communication with a radiation imaging apparatus configured to perform imaging to acquire a radiographic image, the first wireless communication being performed to control the imaging by the radiation imaging apparatus, and a communication device configured to perform second wireless communication with the radiation imaging apparatus to make a setting to be used for the first wireless communication on the radiation imaging apparatus, the method comprising:
performing a first determination whether signal intensity between the communication device and the radiation imaging apparatus exceeds a predetermined threshold; and
performing a second determination whether to perform the second wireless communication with the radiation imaging apparatus based on a predetermined number of times that the signal intensity exceeds the predetermined threshold in the first determination.
